(19) United States
(12) Patent Application Publication
(10) Pub. No.: US 2026/0112498 A1
(43) Pub. Date: Apr. 23, 2026

(54) RISK PREDICTION DEVICE, RISK PREDICTION METHOD, AND RECORDING MEDIUM

(71) Applicant: NEC Corporation, Tokyo (JP)

(72) Inventors: Chenhui HUANG, Tokyo (JP); Kansuke Wagata, Tokyo (JP); Fumiyuki Nihey, Tokyo (JP)

(73) Assignee: NEC Corporation, Tokyo (JP)

(21) Appl. No.: 19/350,495

(22) Filed: Oct. 6, 2025

(30) Foreign Application Priority Data
Oct. 22, 2024 (JP) 2024-185808

(51) Int. Cl. G16H 50/30; G06N 7/01; G06N 20/00

(52) U.S. Cl. CPC G16H 50/30; G06N 7/01; G06N 20/00

(57) ABSTRACT

In the risk prediction device, the acquisition means acquires data of a plurality of different modalities for a single target. In a case where the data of at least one modality among the data of the plurality of modalities is missing, the complementing means acquires the relevance information from the storage unit that stores the relevance information indicating the relevance between the probability distribution data of each modality, and generates the probability distribution data of the missing modality based on the relevance information. The encoder converts the data of each modality into probability distribution data indicating the probability distribution in the latent space. The integration unit integrates the probability distribution data of each modality to generate integrated probability distribution data. The predictor predicts a risk based on the integrated probability distribution data. By using the risk estimation device to estimate disease risk, it is possible to support decision making regarding the subject's lifestyle.

### INCORPORATION BY REFERENCE

This application is based upon and claims the benefit of priority from Japanese Patent Application 2024-185808, filed on Oct. 22, 2024, the disclosure of which is incorporated herein in its entirety by reference.

### TECHNICAL FIELD

The present disclosure relates to risk prediction.

### BACKGROUND ART

A disease risk prediction technique using a machine learning model is known. For example, Patent Document 1 describes a multi-modal machine learning model that predicts the progression of dementia using a plurality of types of input data.
Patent Document 1: International Publication WO 2023/276976

### SUMMARY

In a multi-modal machine learning model, there is such a problem that prediction is hindered in a case where input data of some modalities among a plurality of modalities is missing.

One object of the present disclosure is to provide a risk prediction device capable of highly accurate risk prediction even when input data of some modalities among a plurality of modalities is missing.

According to an example aspect of the present invention, there is provided a risk prediction device comprising:
an acquisition means for acquiring data of a plurality of different modalities for one target;
a storage unit configured to store relevance information indicating relevance between probability distribution data of each modality;
a complementing means configured to generate probability distribution data of a missing modality based on the relevance information in a case where data of at least one modality among the data of the modalities is missing;
an encoder configured to convert data of each modality into probability distribution data indicating a probability distribution in a latent space;
an integration unit configured to integrate the probability distribution data of each modality and generates integrated probability distribution data; and
a predictor configured to predict a risk based on the integrated probability distribution data.

According to another example aspect of the present invention, there is provided a risk prediction method executed by a computer, the method comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.

According to still another example aspect of the present invention, there is provided a program that causes a computer to execute processing comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.

### Effect

According to the present disclosure, it is possible to achieve highly accurate risk prediction even when input data of some modalities among a plurality of modalities is missing.

### BRIEF DESCRIPTION OF THE DRAWINGS

FIG. 1 illustrates an overall configuration of a risk prediction device according to the present disclosure;

FIG. 2 is a block diagram illustrating a hardware configuration of the risk prediction device;

FIG. 3 is a block diagram illustrating a functional configuration of a risk prediction model training device;

FIGS. 4A and 4B are diagrams for explaining expert relevance information;

FIG. 5 is a flowchart of training processing;

FIG. 6 is a block diagram illustrating a functional configuration of the risk prediction device;

FIGS. 7A and 7B are explanatory diagrams of a method of complementing a missing modality;

FIG. 8 is an explanatory diagram of a method of complementing a missing modality;

FIG. 9 is a flowchart of risk prediction processing;

FIG. 10 is a block diagram illustrating another functional configuration of the risk prediction device; and

FIG. 11 is a flowchart of another risk prediction processing.

### EXAMPLE EMBODIMENTS

Hereinafter, preferred example embodiments of the present disclosure will be described with reference to the drawings.

### First Example Embodiment

### Overall Configuration

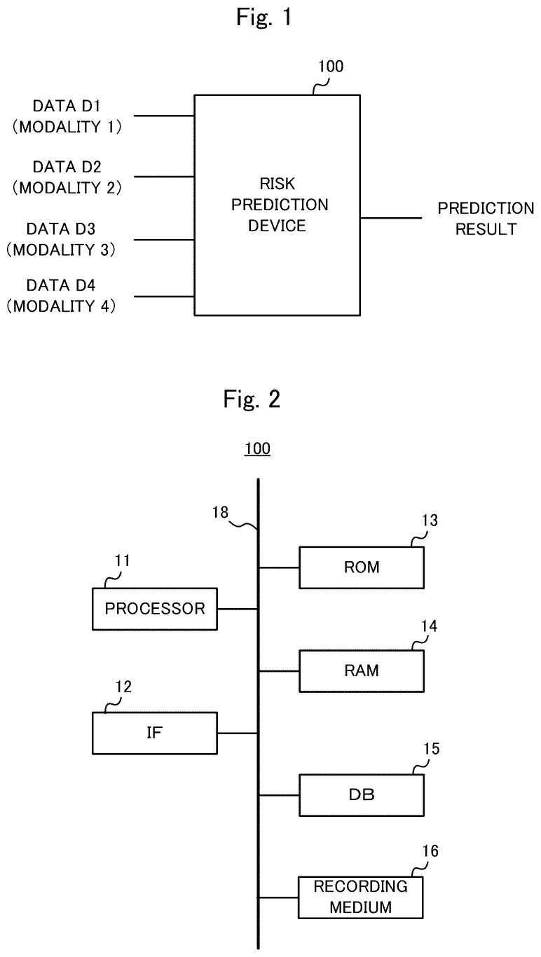

FIG. 1 illustrates an overall configuration of a risk prediction device according to the present disclosure. The risk prediction device 100 predicts a disease risk of a subject based on health data of the subject. Specifically, multimodal data, that is, data of a plurality of different modalities is input to the risk prediction device 100. Note that the term “modality” means a method, means, or the like for expressing information, and the term “multimodal data” means pieces of data in different data formats such as text, image, audio, and sensor data. In the present example embodiment, the multimodal data includes, for example, various pieces of data obtained by health check or the like, such as height, weight, sex, blood pressure, body mass index (BMI), body fat percentage, neutral fat value, smoking status and amount, drinking status and amount, and the like of the subject.

As illustrated in FIG. 1, a plurality of pieces of data (in this example, pieces of data D1 to D4) of different modalities are input to the risk prediction device 100. The risk prediction device 100 converts the input data of each modality into a probability distribution in a latent space, and generates a probability distribution (also referred to as “integrated probability distribution”, “latent representation z”, or the like) obtained by integrating the probability distributions of the modalities. Then, the risk prediction device 100 predicts and outputs the disease risk based on the integrated probability distribution.

At the time of learning, the risk prediction device 100 performs training so that an error between the predicted value of the disease risk obtained based on the integrated probability distribution and the true value of the disease risk prepared in advance as training data becomes small. At the same time, the risk prediction device 100 performs training so that the integrated probability distribution approaches a predetermined reference distribution (for example, normal distribution). When the learning is completed, the risk prediction device 100 stores relevance information indicating the relevance between pieces of probability distribution data obtained at the time of training in a storage unit such as a memory.

On the other hand, at the time of risk prediction, the risk prediction device 100 predicts the disease risk of the subject based on the multimodal data regarding the health of the subject. Here, in a case where data of some modalities among a plurality of modalities is missing, the risk prediction device 100 complements the missing modality data by generating probability distribution data of the modality that is missing (hereinafter also referred to as a “missing modality”) using the relevance information between pieces of probability distribution data stored in the storage unit. Then, the risk prediction device 100 predicts the disease risk of the subject using data of a plurality of modalities including the complemented modality data. As a result, the risk prediction device 100 can predict the disease risk with high accuracy even if data of some modalities is missing.

The risk prediction device 100 can be suitably applied in the medical or healthcare field. For example, the risk prediction device 100 can be used to predict the risk of a lifestyle-related disease based on data obtained in a regular health check.

### [Hardware Configuration]

FIG. 2 is a block diagram illustrating a hardware configuration of the risk prediction device 100. As illustrated in the drawing, the risk prediction device 100 includes a processor 11, an interface (IF) 12, a read only memory (ROM) 13, a random access memory (RAM) 14, a database (DB) 15, and a recording medium 16. These components are connected via a bus 18, for example.

The processor 11 is a computer such as a central processing unit (CPU), and controls the risk prediction device 100 by executing a program prepared in advance. As the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

The processor 11 loads a program stored in the ROM 13 or the recording medium 16 into the RAM 14 and executes each process coded in the program. The processor 11 functions as a part or all of the risk prediction device 100. Specifically, the processor 11 executes training processing and risk prediction processing to be described later.

The IF 12 transmits and receives data to and from an external device. Specifically, in the learning phase, the risk prediction device 100 receives multimodal data on a plurality of persons as training data through the IF 12. Furthermore, in the prediction phase, that is, at the time of risk prediction, the risk prediction device 100 receives the multimodal data of the subject through the IF 12 and outputs a prediction result of the disease risk to the display device or another external device.

The ROM 13 stores various programs executed by the processor 11. The RAM 14 is used as a working memory during execution of various types of processing by the processor 11.

The DB 15 stores various algorithms, data, machine learning models, and the like used when the risk prediction device 100 executes the training processing and risk prediction processing to be described later.

The recording medium 16 is a non-volatile and non-transitory storage medium such as a disk-shaped recording medium or a semiconductor memory. The recording medium 16 may be configured to be detachable from the risk prediction device 100. The recording medium 16 records various programs executed by the processor 11.

In addition to the above, the risk prediction device 100 may include a display device such as a liquid crystal display and an input device such as a keyboard and a mouse. The display and input devices are used by an operator of the risk prediction device 100, for example.

### [Learning Phase]

Next, the learning phase of the risk prediction model will be described.

### (Training Device)

The risk prediction device 100 predicts the disease risk using a trained risk prediction model. Note that, in the following description, the risk prediction model predicts the disease risk from the pieces of data D1 to D4 of four different modalities as an example, but the number of types of data constituting the multimodal data is not limited thereto.

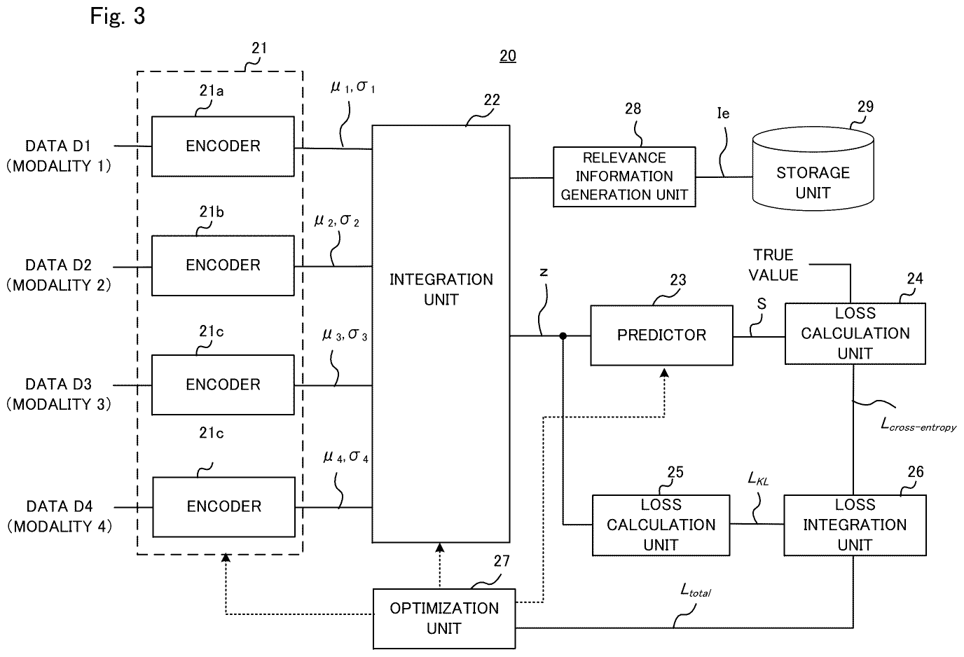

FIG. 3 is a block diagram illustrating a functional configuration of a risk prediction model training device 20. The training device 20 includes an encoder unit 21, an integration unit 22, a predictor 23, loss calculation units 24 and 25, a loss integration unit 26, an optimization unit 27, a relevance information generation unit 28, and a storage unit 29. The encoder unit 21 includes encoders 21a to 21d corresponding to modalities 1 to 4.

The risk prediction model includes the encoder unit 21, the integration unit 22, and the predictor 23. Specifically, a neural network forms the encoder unit 21, the integration unit 22, and the predictor 23. In the learning phase, the training device 20 optimizes the neural network using the training data.

As the training data, multimodal disease risk data for a plurality of persons is prepared. Specifically, the training data is data obtained by collecting attribute data and disease risk values of a plurality of persons. As the attribute data, for example, those having high relevance to the disease risk to be predicted among height, weight, sex, blood pressure, BMI, neutral fat value, blood glucose level, smoking status and amount, drinking status and amount, and the like are used. Note that the disease risk value of each individual corresponds to the correct data in so-called supervised learning, and is hereinafter also referred to as a “true value”. For example, it is assumed that the risk of heart disease is predicted as the disease risk using the blood pressure, BMI, and neutral fat value as the pieces of data D1 to D4. In this case, as the training data, for a plurality of persons, data including blood pressure, BMI, and neutral fat value as the input data and the presence or absence of heart disease as the true value is collected.

In FIG. 3, the pieces of data D1 to D4 of the respective modalities 1 to 4 are input to the encoder unit 21. The data D1 is input to the encoder 21a, the data D2 is input to the encoder 21b, the data D3 is input to the encoder 21c, and the data D4 is input to the encoder 21d. Each of the encoders 21a to 21d projects the input data to a latent space. The “latent space” is an abstract space for expressing information included in the original data in fewer dimensions. In the latent space, essential features and patterns of data are expressed in fewer dimensions. The expression “projects . . . to a latent space” refers to converting the original data into points on the latent space, which is also referred to as “mapping to the latent space”.

Next, each of the encoders 21a to 21d calculates a probability distribution in the latent space for the one of the pieces of input data D1 to D4 of the corresponding modality, and outputs probability distribution data indicating the probability distribution to the integration unit 22. Specifically, the probability distribution data includes an average μ and a standard deviation σ. The probability distribution data (average μ and standard deviation σ) of each modality is also referred to as “expert”.

The integration unit 22 integrates the probability distribution data of each modality and generates the latent representation z as an integrated probability distribution. The latent representation z is expressed by the following formula (1), and is also referred to as an intermediate representation, a hidden representation, a latent variable, or the like.

$$z = μ + σ ε \qquad ( 1 )$$

$$ε ∼ N ( 0 , 1 ) ( N is standard normal distribution )$$

The integration unit 22 outputs the generated latent representation z to the predictor 23 and the loss calculation unit 25.

Note that, as the integration unit 22, for example, the configuration of the product of experts (PoE) layer in the following document can be used. The following document is incorporated herein by reference.

### (Document)

Microbiome-based disease prediction with multimodal variational information bottlenecks, https://doi.org/10.1371/journal.pcbi.1010050

The predictor 23 calculates a disease risk score (hereinafter referred to as a “risk score”) S based on the input latent representation z, and outputs the score S to the loss calculation unit 24.

The loss calculation unit 24 calculates a cross entropy loss L_{cross-entropy} of the risk score S and the true values corresponding to the respective pieces of input data D1 to D4, and outputs the cross entropy loss L_{cross-entropy} to the loss integration unit 26.

The loss calculation unit 25 calculates the similarity between the probability distribution indicated by the latent representation z input from the integration unit 22 and a reference distribution. In a case where the input data D is real number data, a normal distribution is used as the reference distribution. Therefore, the loss calculation unit 25 calculates the Kullback-Leibler (KL) divergence between the probability distribution of each modality and the normal distribution N(0,1) as a loss L_{KL} by the following formula (2) using the average μ and the standard deviation σ indicated by the latent representation z.

$${L}_{K L} = {D}_{K L} [ f ( N ( {μ}_{i} , {σ}_{i} ) \vert \vert f ( N ( 0 , 1 ) ) ] \qquad ( 2 )$$

Note that, in a case where the input data is not real data, the loss calculation unit 25 can use a log-normal distribution, a Poisson distribution, a multinomial logit, an ordinal logit, or the like as the reference distribution according to the format of the input data D.

The loss integration unit 26 calculates a weighted sum of the loss L_{KL} and the loss L_{cross-entropy} by the following formula (3), and outputs the weighted sum to the optimization unit 27 as a total loss Lₜₒₜₐₗ.

$${L}_{total} = {L}_{K L} + λ {L}_{c τ o s s - entropy} \qquad ( 3 )$$

Note that “λ” indicates a weight for weighted addition of the first and second losses.

The optimization unit 27 optimizes the encoder unit 21, the integration unit 22, and the predictor 23 based on the total loss Lₜₒₜₐₗ. Specifically, the optimization unit 27 optimizes the parameters of the neural network forming the encoder unit 21, the integration unit 22, and the predictor 23 so as to reduce the total loss Lₜₒₜₐₗ. Here, since the total loss Lₜₒₜₐₗ is a weighted sum of the loss L_{KL} and the loss L_{cross-entropy}, the optimization unit 27 performs optimization so that the KL divergence between the probability distribution indicated by the latent representation generated by the integration unit 22 and the reference distribution becomes small, that is, the similarity between the probability distribution and the reference distribution becomes high. At the same time, the optimization unit 27 performs optimization so as to reduce the error between the risk score S output by the predictor 23 and the true value.

When the optimization is completed, the relevance information generation unit 28 generates expert relevance information Ie and stores the expert relevance information Ie in the storage unit 29. The expert relevance information is an example of the relevance information, and is information indicating the relevance between experts of respective modalities, that is, respective pieces of probability distribution data (average μ and standard deviation σ). Specifically, the expert relevance information includes average data and covariance data.

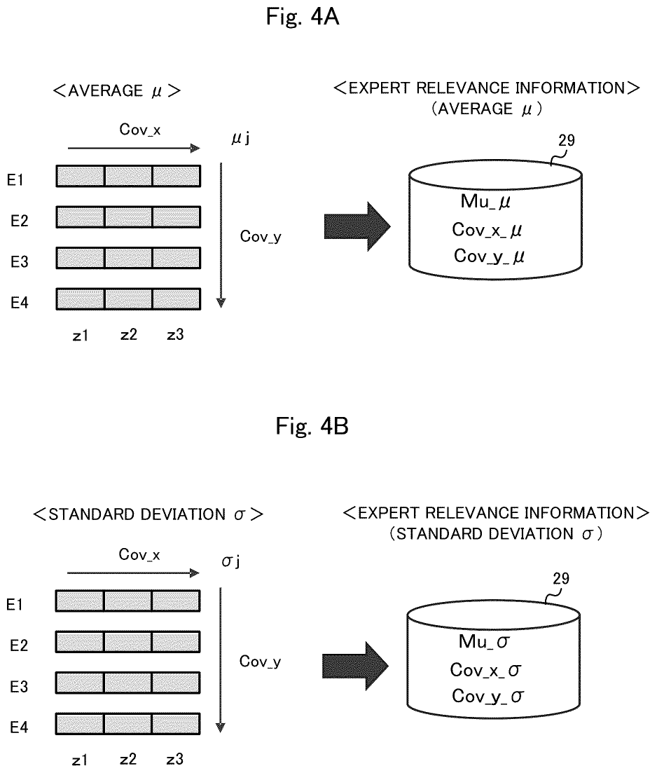

FIGS. 4A and 4B are diagrams for explaining the expert relevance information. Now, assuming that the number of subjects included in the training data is “J”, the training data includes data of four modalities for J subjects. In FIG. 3, experts (each including a pair of an average μ and a standard deviation σ) E1 to E4 corresponding to the respective modalities are output from the encoders 21a to 21d. Now, it is assumed that the latent representation z in the latent space is three-dimensional. An expert for a certain subject j includes an average μj of the subject j and a standard deviation σ j of the subject j. Here, the average μj can be represented by a 4×3 matrix in which the number of dimensions z1 to z3 is taken in the row direction and the experts E1 to E4 are taken in the column direction. Similarly, the standard deviation σ j of the subject j can be represented by a 4×3 matrix in which the number of dimensions z1 to z3 is taken in the row direction and the experts E1 to E4 are taken in the column direction.

The relevance information generation unit 28 first generates expert relevance information for the average μ. Specifically, the relevance information generation unit 28 calculates an average value Mu_μ of the three-dimensional average μ for the J subjects for each of the modalities 1 to 4. Further, the relevance information generation unit 28 calculates the covariance of the average μ in the dimension direction (the x direction in the drawing) and generates a 3×3 covariance matrix Cov_x_μ. Further, the relevance information generation unit 28 calculates the covariance in the modality direction (the y direction in the drawing) of the average μ, and generates a 4×4 covariance matrix Cov_y_μ. Then, the relevance information generation unit 28 stores the obtained average value Mu_μ, covariance matrix Cov_x_μ, and covariance matrix Cov_y_μ in the storage unit 29 as expert relevance information for the average μ.

Similarly, the relevance information generation unit 28 generates expert relevance information for the standard deviation σ. Specifically, the relevance information generation unit 28 calculates, for each of the modalities 1 to 4, an average value Mu_σ of the three-dimensional standard deviation σ for the J subjects. In addition, the relevance information generation unit 28 calculates the covariance of the standard deviation σ in the dimension direction (the x direction in the drawing) and generates a 3×3 covariance matrix Cov_x_σ. Furthermore, the relevance information generation unit 28 calculates the covariance of the standard deviation σ in the modality direction (the y direction in the drawing) and generates a 4×4 covariance matrix Cov_y_σ. Then, the relevance information generation unit 28 stores the obtained average value Mu_σ, covariance matrix Cov_x_σ, and covariance matrix Cov_y_σ in the storage unit 29 as expert relevance information for the standard deviation σ.

As described above, by storing the expert relevance information indicating the relevance between the experts in the storage unit 29 when the learning is completed, even if data of some modalities is missing in the inference phase, the data can be complemented, as will be described later.

### (Training Processing)

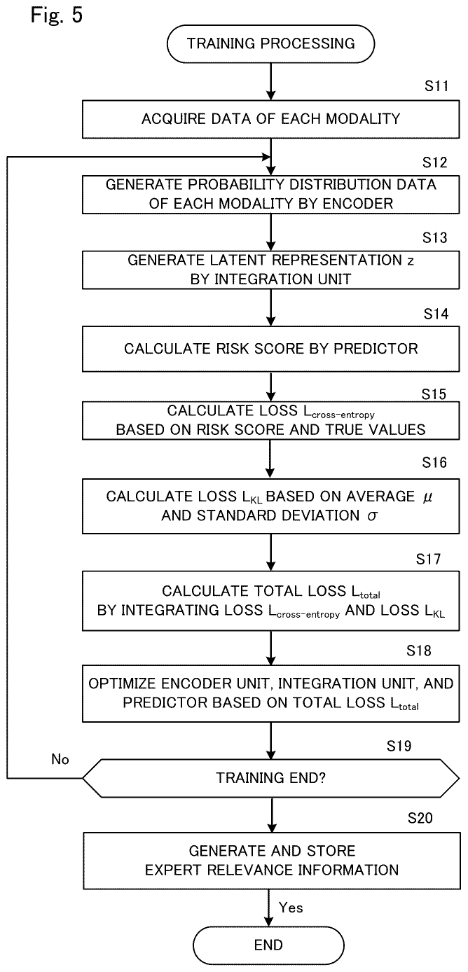

Next, the training processing performed by the above training device 20 will be described. FIG. 5 is a flowchart of the training processing. This processing is achieved by the processor 11 illustrated in FIG. 2 executing a program prepared in advance and operating as each component illustrated in FIG. 3.

First, the encoder unit 21 acquires data of each modality included in the training data (step S11). Next, the encoder unit 21 projects each data to a latent space by each of the encoders 21a to 21d to generate an expert (a pair of the average μ and the standard deviation σ) of each modality (step S12). Next, the integration unit 22 integrates experts of the respective modalities to generate the latent representation z in the latent space (step S13). Next, the predictor 23 calculates a risk score S based on the latent representation z (step S14).

Next, the loss calculation unit 24 calculates the loss L_{cross-entropy} based on the risk score S and the true value (step S15). In addition, the loss calculation unit 25 calculates the loss L_{KL} using the average μ and the standard deviation σ of each modality (step S16). Next, the loss integration unit 26 calculates the total loss Lₜₒₜₐₗ from the loss L_{cross-entropy} and the loss L_{KL} (step S17). Next, the optimization unit 27 optimizes the parameters of the encoder unit 21, the integration unit 22, and the predictor 23 based on the total loss Lₜₒₜₐₗ (step S18).

Next, the training device 20 determines whether or not a predetermined training end condition has been satisfied (step S19). Examples of the training end condition include that a predetermined number of pieces of attribute data prepared as training data has been used, the total loss has become equal to or less than a predetermined value, and the total loss has converged. If the training end condition is not satisfied (step S19: No), the process returns to step S12.

On the other hand, when the training end condition is satisfied (step S19: Yes), the relevance information generation unit 28 generates the expert relevance information Ie indicating the relevance between the experts of the respective modalities as described above, and stores the expert relevance information Ie in the storage unit 29 (step S20). Then, the training processing ends.

### [Prediction Phase]

Next, the prediction phase by the risk prediction device will be described. In the prediction phase, the risk prediction device 100 predicts the disease risk of a certain subject based on multimodal data of the subject. At this time, the risk prediction device 100 uses the risk prediction model trained in the learning phase, specifically, the encoder unit 21, the integration unit 22, and the predictor 23. Furthermore, in a case where some modalities of multimodal data of a certain subject are missing in the prediction phase, the risk prediction device 100 predicts the risk after complementing the data of the missing modalities.

### (Risk Prediction Device)

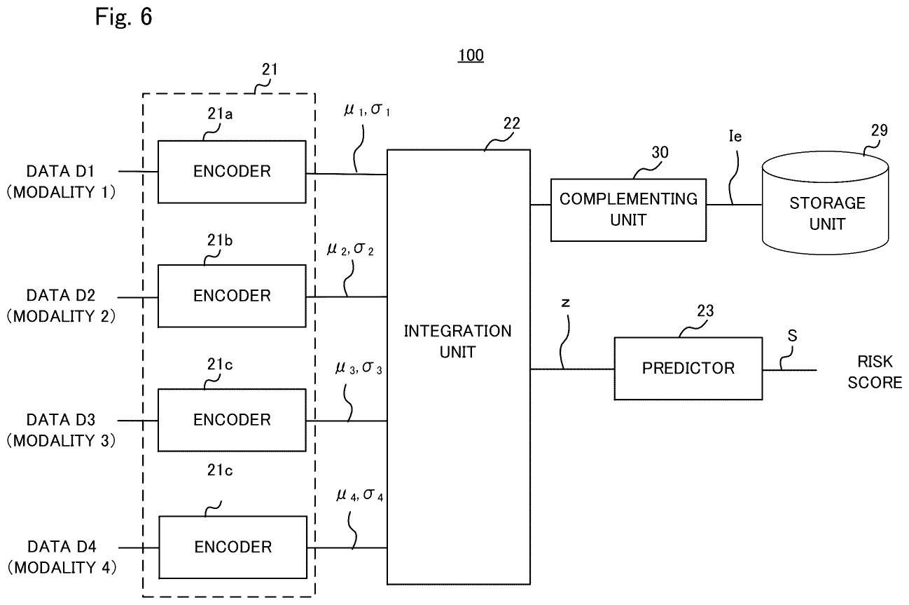

FIG. 6 is a block diagram illustrating a functional configuration of the risk prediction device. The risk prediction device 100 includes the encoder unit 21, the integration unit 22, and the predictor 23 optimized in the learning phase. In addition, the risk prediction device 100 includes a complementing unit 30 for complementing data of the missing modality.

(I) in a Case where there is No Missing Modality

First, a case where there is no missing modality in the input data will be described. In this case, pieces of data D1 to D4 of four different modalities are input to the encoder unit 21 for a certain subject. Each of the encoders 21a to 21d projects the corresponding one of the input data D1 to D4 to a latent space, generates probability distribution data (expert) including the average μ and the standard deviation σ, and outputs the probability distribution data to the integration unit 22.

The integration unit 22 integrates the probability distribution data of each modality, and generates the latent representation z as an integrated probability distribution obtained by integrating the probability distributions of the modalities. The integration unit 22 outputs the latent representation z to the predictor 23.

The predictor 23 calculates and outputs the risk score S indicating a disease risk based on the input latent representation z. In this way, the disease risk of the subject can be predicted based on the multimodal data.

(II) in a Case where there is Missing Modality

Next, a case where there is a missing modality in the input data will be described. In this case, the complementing unit 30 generates data of the missing modality using the expert relevance information Ie stored in the storage unit 29. Hereinafter, a modality that is missing is referred to as a “missing modality”, and a modality that is not missing is referred to as a “non-missing modality”.

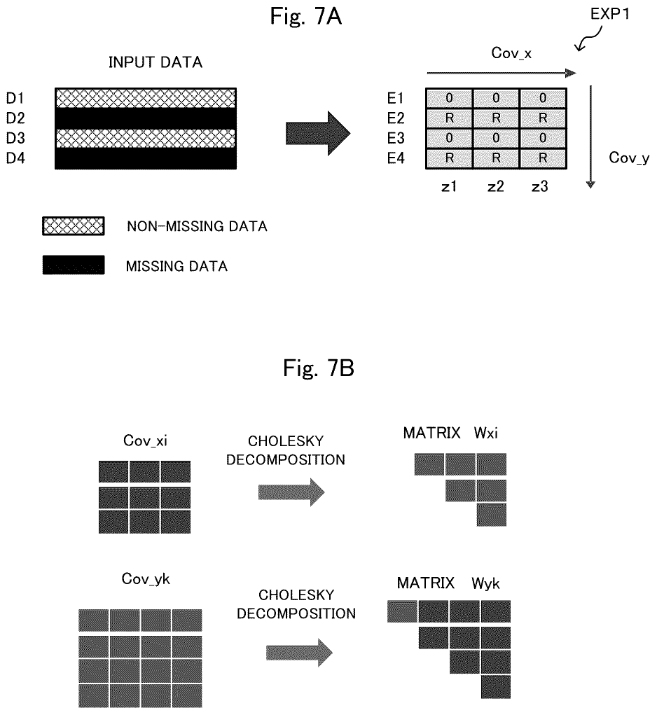

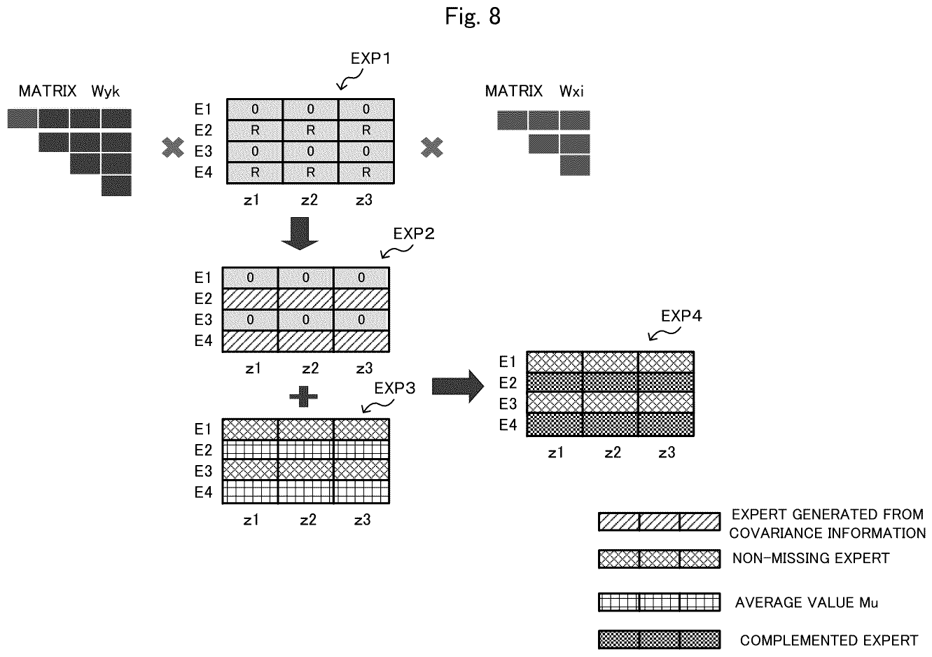

An example of complementing the missing modality will be described. FIGS. 7A to 8 are explanatory diagrams of a method of complementing the missing modality. Here, it is assumed that data of the modalities 2 and 4 are missing among the modalities 1 to 4. In the input data of FIG. 7A, the pieces of data D2 and D4 of the missing modalities 2 and 4 are illustrated in black, and the pieces of data D1 and D3 of the non-missing modalities 1 and 3 are illustrated in gray.

The complementing unit 30 generates a matrix EXP1 including experts of the modalities 1 to 4. Hereinafter, an expert of a missing modality is also referred to as a “missing expert”, and an expert of a non-missing modality is also referred to as a “non-missing expert”.

The complementing unit 30 first inserts a random number that follows a normal distribution into the row of the missing expert. In addition, the complementing unit 30 inserts “0” into all the rows of non-missing experts. In this way, the complementing unit 30 generates the matrix EXP1 based on the input data. In FIGS. 7A, the cells having random numbers in the matrix EXP1 are indicated by “R”.

Next, the complementing unit 30 acquires the expert relevance information Ie corresponding to the missing modality from the storage unit 29. In this example, since the missing modalities are the modalities 2 and 4, the complementing unit 30 obtains covariance matrices Cov_x and Cov_y corresponding to the missing modalities 2 and 4 from the storage unit 29. Since the data of each modality includes the average μ and the standard deviation σ, the complementing unit 30 acquires the covariance matrices Cov_x_μ and Cov_y_μ related to the average μ, and the covariance matrix Cov_x_σ in the dimension direction and the covariance matrix Cov_y_σ in the modality direction related to the standard deviation. Since the method of complementing the average μ included in the missing expert and the method of complementing the standard deviation σ are the same, the distinction between the average μ and the standard deviation σ is omitted below for convenience of description. That is, the complementing unit 30 acquires the covariance matrix Cov_xi in the dimension direction and the covariance matrix Cov_yk in the modality direction with respect to the average μ and the standard deviation σ.

Next, as illustrated in FIG. 7B, the complementing unit 30 performs Cholesky decomposition on the obtained covariance matrix Cov_xi to generate a matrix Wxi, and performs Cholesky decomposition on the covariance matrix Cov_yk to generate a matrix Wyk, in order to achieve, for example, improved efficiency of the matrix operation.

Next, the complementing unit 30 multiplies the matrix EXP1 by the matrices Wyk and Wxi in the order illustrated in FIG. 8 to obtain a matrix EXP2 incorporating covariance. In the matrix EXP2, the rows of the non-missing experts E1 and E3 are “0”, and the values of the missing experts E2 and E4 are values corresponding to the changes in the experts generated from the covariance matrices.

Next, the complementing unit 30 generates a matrix EXP3 into which the non-missing experts are inserted. Specifically, the complementing unit 30 puts the values of the non-missing experts, that is, the values of the experts calculated from the input data into the rows of the non-missing modalities 1 and 3. In addition, the complementing unit 30 acquires the averages Mu_μ and Mu_σ corresponding to the missing modalities 2 and 4 from the storage unit 29. Then, the complementing unit 30 puts the averages Mu_μ into the rows of the missing experts E2 and E4 in the matrix EXP3 for the average μ. In addition, the complementing unit 30 puts the averages Mu_σ into the rows of the missing experts E2 and E4 in the matrix EXP3 for the standard deviation σ. Thus, in the matrix EXP3, the experts calculated from the input data are put into the rows E1 and E3 of the non-missing experts, and the values of the averages Mu stored in the storage unit 29 in the learning phase are put into the rows of the missing experts E2 and E4.

Next, the complementing unit 30 adds the matrices EXP2 and EXP3 together to obtain a matrix EXP4. In the matrix EXP4 obtained in this way, the rows of the non-missing experts E1 and E3 include the experts calculated from the input data, and the rows of the missing experts E2 and E4 include experts generated by adding the changes calculated based on the covariance matrices to the values of the averages Mu. In this manner, the complementing unit 30 can complement the experts of the missing modalities using the expert relevance information Ie stored in the storage unit 29 in the learning phase.

As described above, when the experts of the missing modalities are complemented, the risk prediction device 100 predicts a disease risk of the subject using complemented experts. The processing of the risk prediction device 100 after the missing experts are complemented is similar to the processing in a case where there is no missing expert in the input data.

### (Risk Prediction Processing)

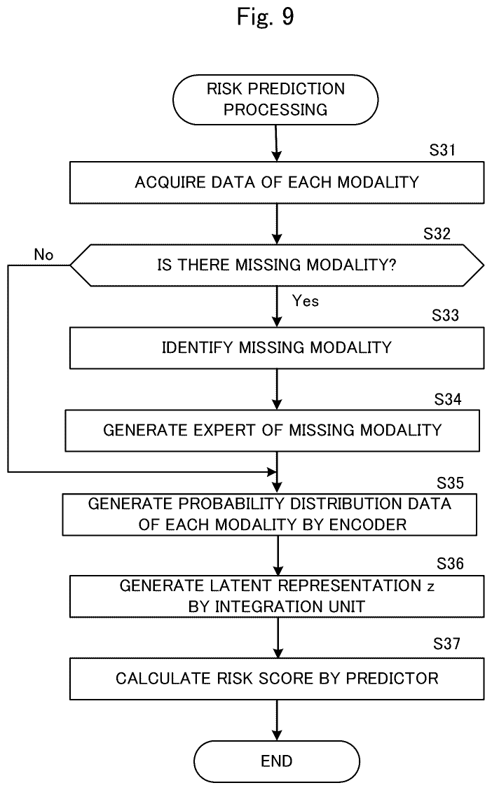

Next, risk prediction processing executed by the risk prediction device 100 will be described. FIG. 9 is a flowchart of the risk prediction processing. This processing is achieved by the processor 11 illustrated in FIG. 2 executing a program prepared in advance and operating as each element illustrated in FIG. 6.

First, the risk prediction device 100 acquires input data of each modality for the subject (step S31). Next, the risk prediction device 100 determines whether there is a missing modality in the input data (step S32). If there is no missing modality (step S32: No), the processing proceeds to step S35. On the other hand, if there is a missing modality (step S32: Yes), the risk prediction device 100 identifies the missing modality (step S33), and generates an expert of the missing modality by the complementing unit 30 (step S34).

Next, the encoder unit 21 generates an expert of each modality from the input data of the non-missing modalities (step S35). Next, the integration unit 22 integrates the experts of the respective modalities, specifically, the generated experts of the non-missing modalities and the experts of the missing modality obtained by the complementing processing to generate the latent representation z in a latent space (step S36). Next, the predictor 23 calculates the risk score S based on the latent representation z and outputs the risk score S (step S37). Then, the risk prediction processing ends.

### [Modification]

In the first example embodiment described above, the risk prediction device is applied to generate attribute data on human health, but the application of the present disclosure is not limited thereto. For example, the present disclosure may be applied to inspection and diagnosis of machines and devices. That is, the method of the present disclosure may be applied to estimate the state of the machine or device based on data of a plurality of modalities detected and collected in inspection or diagnosis.

### Second Example Embodiment

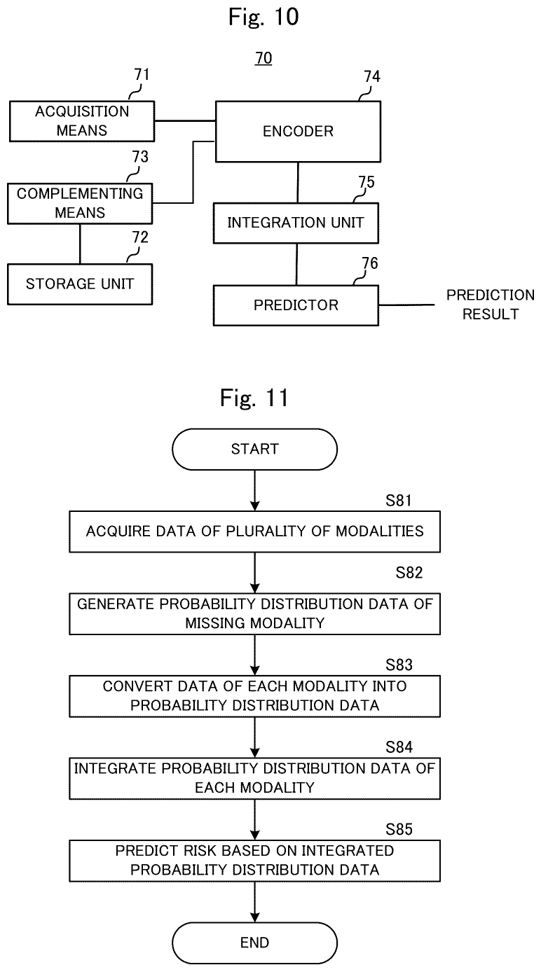

FIG. 10 is a block diagram illustrating a functional configuration of a risk prediction device of a second example embodiment. The risk prediction device 70 includes an acquisition means 71, a storage unit 72, a complementing means 73, an encoder 74, an integration unit 75, and a predictor 76.

FIG. 11 is a flowchart of processing by the risk prediction device according to the second example embodiment. The acquisition means 71 acquires data of a plurality of different modalities for a single target such as a subject (step S81). In a case where the data of at least one modality among the data of the plurality of modalities is missing, the complementing means 73 acquires the relevance information from the storage unit 72 that stores the relevance information indicating the relevance between the probability distribution data of each modality, and generates the probability distribution data of the missing modality based on the relevance information (step S82). The encoder 74 converts the data of each modality into probability distribution data indicating the probability distribution in the latent space (step S83). The integration unit 75 integrates the probability distribution data of each modality to generate integrated probability distribution data (step S84). The predictor 76 predicts a risk based on the integrated probability distribution data (step S85).

According to the risk prediction device 70 of the second example embodiment, it is possible to predict a risk with high accuracy even when there is a missing part in the input data.

A part or all of the example embodiments described above may also be described as the following supplementary notes, but not limited thereto.

### Supplementary Note 1

A risk prediction device comprising:
an acquisition means configured to acquire data of a plurality of different modalities for one target;
a storage unit configured to store relevance information indicating relevance between probability distribution data of each modality;
a complementing means configured to generate probability distribution data of a missing modality based on the relevance information in a case where data of at least one modality among the data of the modalities is missing;
an encoder configured to convert data of each modality into probability distribution data indicating a probability distribution in a latent space;
an integration unit configured to integrate the probability distribution data of each modality and generates integrated probability distribution data; and
a predictor configured to predict a risk based on the integrated probability distribution data.

### Supplementary Note 2

The risk prediction device according to Supplementary note 1, wherein the relevance information includes a covariance between probability distribution data of each modality.

### Supplementary Note 3

The risk prediction device according to Supplementary note 2, wherein the complementing means generates the probability distribution data of the missing modality based on a random number and the relevance information.

### Supplementary Note 4

The risk prediction device according to Supplementary note 1, further comprising a relevance information generation means configured to generate the relevance information based on data of a plurality of different modalities for a plurality of targets.

### Supplementary Note 5

The risk prediction device according to Supplementary note 1,
wherein the probability distribution data includes an average and a standard deviation, and
wherein the relevance information includes a covariance of the average of each modality and a covariance of the standard deviation of each modality.

### Supplementary Note 6

The risk prediction device according to Supplementary note 4, further comprising a training means configured to optimize the encoder, the integration unit, and the predictor based on a first loss indicating similarity between a probability distribution corresponding to each modality and a predetermined reference distribution and a second loss indicating an error between a prediction result by the predictor and a true value prepared in advance.

### Supplementary Note 7

The risk prediction device according to Supplementary note 6, wherein the relevance information generation means generates the relevance information using the encoder, the integration unit, and the predictor after optimization by the training means.

### Supplementary Note 8

The risk prediction device according to Supplementary note 1, wherein the predictor predicts a disease risk of the target based on data of a plurality of modalities related to health of the target by a trained machine learning model.

### Supplementary Note 9

A risk prediction method executed by a computer, the method comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.

### Supplementary Note 10

A program that causes a computer to execute processing comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Processor
20 Prediction model training device
21 Encoder unit
21a-21d Encoder
22 Integration unit
23 Predictor
24, 25 Loss calculation unit
26 Loss integration unit
27 Optimization unit
100 Risk prediction device

1. A risk prediction device comprising:
a storage configured to store relevance information indicating relevance between probability distribution data of each modality;
at least one memory configured to store instructions; and
at least one processor configured to execute the instructions to:
acquire data of a plurality of different modalities for one target;
generate probability distribution data of a missing modality based on the relevance information in a case where data of at least one modality among the data of the modalities is missing;
convert data of each modality into probability distribution data indicating a probability distribution in a latent space;
integrate the probability distribution data of each modality and generates integrated probability distribution data; and
predict a risk based on the integrated probability distribution data.

2. The risk prediction device according to claim 1, wherein the relevance information includes a covariance between probability distribution data of each modality.

3. The risk prediction device according to claim 2, wherein the processor generates the probability distribution data of the missing modality based on a random number and the relevance information.

4. The risk prediction device according to claim 1, wherein the processor is further configured to execute the instructions to generate the relevance information based on data of a plurality of different modalities for a plurality of targets.

5. The risk prediction device according to claim 1,
wherein the probability distribution data includes an average and a standard deviation, and
wherein the relevance information includes a covariance of the average of each modality and a covariance of the standard deviation of each modality.

6. The risk prediction device according to claim 4, wherein the processor is further configured to execute the instructions to optimize the encoder, the integration unit, and the predictor based on a first loss indicating similarity between a probability distribution corresponding to each modality and a predetermined reference distribution and a second loss indicating an error between a prediction result by the predictor and a true value prepared in advance.

7. The risk prediction device according to claim 6, wherein the processor generates the relevance information using the encoder, the integration unit, and the predictor after optimization by the training means.

8. The risk prediction device according to claim 1, wherein the processor predicts a disease risk of the target based on data of a plurality of modalities related to health of the target by a trained machine learning model.

9. A risk prediction method executed by a computer, comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.

10. A non-transitory computer-readable medium storing a program, the program causing a computer to execute processing comprising:
acquiring data of a plurality of different modalities for one target;
in a case where data of at least one modality among the modalities is missing, acquiring relevance information indicating relevance between probability distribution data of each modality from a storage unit and generating probability distribution data of the missing modality based on the relevance information;
converting data of each modality into probability distribution data indicating a probability distribution in a latent space;
integrating the probability distribution data of each modality and generating integrated probability distribution data; and
predicting a risk based on the integrated probability distribution data.
